(19) United States
(12) Patent Application Publication
(10) Pub. No.: US 2026/0327189 A1
(43) Pub. Date: Oct. 22, 2026

(54) DIMETHYLTRYPTAMINE ANALOGUES AS NITRIC OXIDE DELIVERY DRUGS

(71) Applicants: Robert B. PERNI, Marlborough, MA (US); Glenn SHORT, Scituate, MA (US); Tanweer A. KHAN, Bridgewater, NJ (US)

(72) Inventors: Robert B. PERNI, Marlborough, MA (US); Glenn SHORT, Scituate, MA (US); Tanweer A. KHAN, Bridgewater, NJ (US)

(21) Appl. No.: 19/571,951

(22) Filed: Mar. 19, 2026

Related U.S. Application Data
(63) Continuation of application No. 18/657,306, filed on May 7, 2024, now Pat. No. 12,606,525.
Application No. 18/657,306 is a continuation of application No. 18/147,499, filed on Dec. 28, 2022, now Pat. No. 12,012,381.
(60) Provisional application No. 63/295,199, filed on Dec. 30, 2021.

(51) Int. Cl. C07D 209/16

(52) U.S. Cl. CPC C07D 209/16; C07B 2200/05

(57) ABSTRACT

Provided herein are compounds of Formula (I), (II), or a pharmaceutically acceptable salt thereof. Also provided herein are pharmaceutical compositions comprising a compound of Formula (I), (II), or pharmaceutically acceptable salt thereof, and methods of using a compound of Formula (I), (II), or a pharmaceutically acceptable salt thereof.

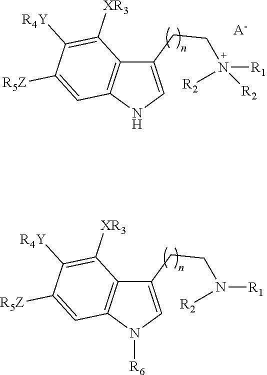

### CROSS-REFERENCE TO RELATED APPLICATION

This application is a continuation of U.S. patent application Ser. No. 18/657,306, filed May 7, 2024, which is a continuation of U.S. patent application Ser. No. 18/147,499, filed Dec. 28, 2022, now U.S. Pat. No. 12,012,381, which claims the benefit of priority to U.S. Provisional Application No. 63/295,199, filed Dec. 30, 2021, each of which is hereby incorporated by reference in their entirety.

### BRIEF SUMMARY OF THE INVENTION

In embodiments, the present disclosure provides dimethyltryptamine derivatives that release nitric oxide (NO) in vivo.

In embodiments, the present disclosure provides prodrugs of dimethyltryptamine and derivatives thereof.

In embodiments, the present disclosure provides compounds of Formula (I), Formula (II), or pharmaceutically acceptable salts thereof.

In embodiments, the present disclosure provides a compound of Formula (I):

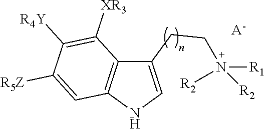

or a pharmaceutically acceptable salt thereof; wherein,
R¹, R³, R⁴ and R⁵ are independently H, halogen, C₁-C₆ alkyl, —(C═O)(CR₇R₇′)ₙ—ONO₂, or —(C═O)(CR₇R₇′)ₘ—CH(NH₂)CH₂ONO₂;
each R² is independently C₁-C₆ alkyl;
X, Y, and Z are independently absent, H, O, S, NH and —O—(P═O)OHO—;
R⁷ and R⁷′ are independently H, halogen, or C₁-C₆ alkyl;
m is 2, 3, or 4;
each n is independently 1, 2, 3, 4 or 5; and
A⁻ is a pharmaceutically acceptable anion,
wherein at least one of R₁, R₃, R₄ and R₅ are —(C—O)(CR₇R₇′)ₙ—ONO₂ or —(C═O)(CR₇R₇′)ₘ—CH(NH₂)CH₂ONO₂.

In embodiments, the present disclosure provides a compound of Formula (II):

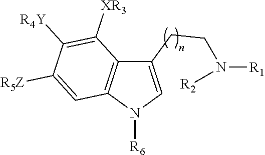

or a pharmaceutically acceptable salt thereof; wherein,
R₃, R₄, and R₅ are independently H, halogen, C₁-C₆ alkyl, —(C—O)(CR₇R₇′)ₙ—ONO₂, —(C═O)(CR₇R₇′)ₘ—CH(NH₂)CH₂ONO₂;
R₁ and R₆ are independently H, C₁-C₆ alkyl, —(C—O)(CH₂)ₗ—ONO₂;
—(C═O)(CH₂)ₘ—CH(NH₂)CH₂ONO₂;
R₂ is H or C₁-C₆ alkyl;
R₇ and R₇′ are independently H, halogen, or C₁-C₆ alkyl;
X, Y, and Z are independently absent, O, S, NH and —O—(P═O)OHO—,
m is 2, 3, or 4; and
each n is independently 1, 2, 3, 4 or 5;
wherein at least one of R₁, R₃, R₄, R₅ and R₆ is —(C═O)(CR₇R₇′)ₙ—ONO₂, —(CO)(CR₇R₇′)ₘ—CH(NH₂)CH₂ONO₂.

In embodiments, the present disclosure provides a pharmaceutical composition, comprising a compound of the present disclosure (e.g., compounds of Formula (I), (II) or Table 1) and a pharmaceutically acceptable excipient.

In embodiments, the present disclosure provides methods of using one or more compounds of the present disclosure (e.g., compounds of Formula (I), (II) or Table 1), e.g., as NO delivery agents.

### DETAILED DESCRIPTION OF THE INVENTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to administering a compound or pharmaceutically acceptable salt of the compound or a composition or formulation comprising the compound or pharmaceutically acceptable salt of the compound to a patient.

The term “treating” as used herein with regard to a patient or subject, refers to improving at least one symptom of the patient's or subject's disorder. In some embodiments, treating can be improving, or at least partially ameliorating a disorder or one or more symptoms of a disorder.

The term “therapeutically effective” applied to dose or amount refers to that quantity of a compound or pharmaceutical formulation that is sufficient to result in a desired clinical benefit after administration to a patient or subject in need thereof.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. The acids that may be used to prepare pharmaceutically acceptable acid addition salts of such basic compounds are those that form non-toxic acid addition salts, i.e., salts containing pharmaceutically acceptable anions, including but not limited to malate, oxalate, chloride, bromide, iodide, nitrate, acetate, tartrate, oleate, fumarate, formate, benzoate, glutamate, methanesulfonate, benzenesulfonate, and p-toluenesulfonate salts. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

The term “substituted” used herein means any of the groups described herein (e.g., alkyl, alkenyl, alkynyl, alkoxy, aryl, cycloalkyl, cycloalkenyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

### Compounds

In embodiments, the present disclosure provides dimethyltryptamine derivatives that release nitric oxide (NO) in vivo. In embodiments, the present disclosure provides prodrugs of dimethyltryptamine and derivatives thereof. In embodiments, the present disclosure provides compound of Formula (I) and (II), or pharmaceutically acceptable salts thereof.

In embodiments, the present disclosure provides a compound of Formula (I):

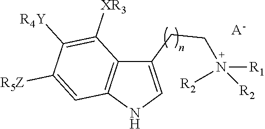

or a pharmaceutically acceptable salt thereof; wherein,
R₁, R₃, R₄ and R₅ are independently H, halogen, C₁-C₆ alkyl, —(C—O)(CR₇R₇′)ₙ—ONO₂, or —(C═O)(CR₇R₇′)ₘ—CH(NH₂)CH₂ONO₂;
R₂ is independently C₁-C₆ alkyl;
X, Y, and Z are independently absent, H, O, S, NH and —O—(P═O)OHO—;
m is 2, 3, or 4;
each n is independently 1, 2, 3, 4 or 5; and
A⁻ is a pharmaceutically acceptable anion,
wherein at least one of R₁, R₃, R₄ and R₅ are —(C═O)(CR₇R₇′)ₙ—ONO₂ or —(C—O)(CR₇R₇′)ₘ—CH(NH₂)CH₂ONO₂.

In embodiments of the compounds of Formula (I), R₁ is C₁-C₆ alkyl. In embodiments of the compounds of Formula (I), R₁ is methyl, ethyl, or propyl. In embodiments of the compounds of Formula (I), R₁ is methyl. In embodiments of the compounds of Formula (I), R₁ is H.

In embodiments of the compounds of Formula (I), R₁ is C₁-C₆ alkyl, —(C—O)(CR₇R₇′)ₙ—ONO₂, or —(C═O)(CR₇R₇′)ₘ—CH(NH₂)CH₂ONO₂. In embodiments of the compounds of Formula (I), R₁ is H, —(C═O)(CR₇R₇′)ₙ—ONO₂, or —(C═O)(CR₇R₇′)ₘ—CH(NH₂)CH₂ONO₂. In embodiments of the compounds of Formula (I), R₁ is H, C₁-C₆ alkyl, or —(C═O)(CR₇R₇′)ₘ—CH(NH₂)CH₂ONO₂. In embodiments of the compounds of Formula (I), R₁ is H, C₁-C₆ alkyl, or —(C—O)(CR₇R₇′)ₙ—ONO₂. In embodiments of the compounds of Formula (I), R₁ is —(C—O)(CR₇R₇′)ₙ—ONO₂ or —(C═O)(CR₇R₇′)ₘ—CH(NH₂)CH₂ONO₂. In embodiments of the compounds of Formula (I), R₁ is C₁-C₆ alkyl or —(CO)(CR₇R₇′)ₘ—CH(NH₂)CH₂ONO₂. In embodiments of the compounds of Formula (I), R₁ is halogen, C₁-C₆ alkyl, —(C═O)(CR₇R₇′)ₙ—ONO₂, or —(C—O)(CR₇R₇′)ₘ—CH(NH₂)CH₂ONO₂.

In embodiments of the compounds of Formula (I), R₁ is —(C═O)(CR₇R₇′)ₙ—ONO₂. In embodiments of the compounds of Formula (I), R₁ is —(C═O)(CR₇R₇′)ₘ—CH(NH₂)CH₂ONO₂. In embodiments of the compounds of Formula (I), R₁ is —(C—O)(CH₂)ₙ—ONO₂. In embodiments of the compounds of Formula (I), R₁ is —(C═O)(CH₂)ₘ—CH(NH₂)CH₂ONO₂. In embodiments of the compounds of Formula (I), R₁ is —(C═O)(CH₂)₃—ONO₂. In embodiments of the compounds of Formula (I), R₁ is —(C═O)(CH₂) 2-CH(NH₂)CH₂ONO₂.

In embodiments of the compounds of Formula (I), R₃ is H. In embodiments of the compounds of Formula (I), R₃ is halogen. In embodiments of the compounds of Formula (I), R₃ is C₁-C₆ alkyl.

In embodiments of the compounds of Formula (I), R₃ is H, C₁-C₆ alkyl, —(C═O)(CR₇R₇′)ₙ—ONO₂, —(C═O)(CR₇R₇′)ₘ—CH(NH₂)CH₂ONO₂. In embodiments of the compounds of Formula (I), R₃ is H, halogen, —(C═O)(CR₇R₇′)ₙ—ONO₂, —(C═O)(CR₇R₇′)ₘ—CH(NH₂)CH₂ONO₂. In embodiments of the compounds of Formula (I), R₃ is H, halogen, C₁-C₆ alkyl, or —(C═O)(CR₇R₇′)ₘ—CH(NH₂)CH₂ONO₂. In embodiments of the compounds of Formula (I), R₃ is H, halogen, C₁-C₆ alkyl, or —(C—O)(CR₇R₇′)ₙ—ONO₂. In embodiments of the compounds of Formula (I), R₃ is H, halogen, or C₁-C₆ alkyl. In embodiments of the compounds of Formula (I), R₃ is H or halogen. In embodiments of the compounds of Formula (I), R₃ is H or C₁-C₆ alkyl. In embodiments of the compounds of Formula (I), R₃ is halogen or C₁-C₆ alkyl.

In embodiments of the compounds of Formula (I), R₃ is —(C═O)(CR₇R₇′)ₙ—ONO₂ or —(CO)(CR₇R₇′)ₘ—CH(NH₂)CH₂ONO₂. In embodiments of the compounds of Formula (I), R₃ is —(C═O)(CR₇R₇′)ₙ—ONO₂. In embodiments of the compounds of Formula (I), R₃ is —(C—O)(CR₇R₇′)ₘ—CH(NH₂)CH₂ONO₂.

In embodiments of the compounds of Formula (I), R₄ is H, halogen, or C₁-C₆ alkyl. In embodiments of the compounds of Formula (I), R₄ is H or halogen. In embodiments of the compounds of Formula (I), R₁ is H or C₁-C₆ alkyl. In embodiments of the compounds of Formula (I), R₄ is halogen or C₁-C₆ alkyl.

In embodiments of the compounds of Formula (I), R₄ is halogen, C₁-C₆ alkyl, —(C═O)(CR₇R₇′)ₙ—ONO₂, —(C═O)(CR₇R₇′)ₘ—CH(NH₂)CH₂ONO₂. In embodiments of the compounds of Formula (I), R₄ is H, C₁-C₆ alkyl, —(C═O)(CR₇R₇′)ₙ—ONO₂, —(C═O)(CR₇R₇′)ₘ—CH(NH₂)CH₂ONO₂. In embodiments of the compounds of Formula (I), R₄ is H, halogen, —(C═O)(CR₇R₇′)ₙ—ONO₂, —(C═O)(CR₇R₇′)ₘ—CH(NH₂)CH₂ONO₂. In embodiments of the compounds of Formula (I), R₄ is H, halogen, C₁-C₆ alkyl, or —(C═O)(CR₇R₇′)ₘ—CH(NH₂)CH₂ONO₂. In embodiments of the compounds of Formula (I), R₄ is H, halogen, C₁-C₆ alkyl, or —(C═O)(CR₇R₇′)ₙ—ONO₂.

In embodiments of the compounds of Formula (I), R₄ is —(C═O)(CR₇R₇′)ₙ—ONO₂ or —(C—O)(CR₇R₇′)ₘ—CH(NH₂)CH₂ONO₂. In embodiments of the compounds of Formula (I), R₄ is —(C═O)(CR₇R₇′)ₙ—ONO₂. In embodiments of the compounds of Formula (I), R₄ is —(C═O)(CR₇R₇′)ₘ—CH(NH₂)CH₂ONO₂.

In embodiments of the compounds of Formula (I), R₅ is H. In embodiments of the compounds of Formula (I), R₅ is halogen. In embodiments of the compounds of Formula (I), R₅ is C₁-C₆ alkyl. In embodiments of the compounds of Formula (I), R₅ is H, halogen, or C₁-C₆ alkyl. In embodiments of the compounds of Formula (I), R₅ is H or halogen. In embodiments of the compounds of Formula (I), R₅ is H or C₁-C₆ alkyl. In embodiments of the compounds of Formula (I), R₅ is halogen or C₁-C₆ alkyl.

In embodiments of the compounds of Formula (I), R₅ is halogen, C₁-C₆ alkyl, —(C═O)(CR₇R₇′)ₙ—ONO₂, —(C—O)(CR₇R₇′)ₘ—CH(NH₂)CH₂ONO₂. In embodiments of the compounds of Formula (I), R₅ is H, C₁-C₆ alkyl, —(C═O)(CR₇R₇′)ₙ—ONO₂, —(C═O)(CR₇R₇′)ₘ—CH(NH₂)CH₂ONO₂. In embodiments of the compounds of Formula (I), R₅ is H, halogen, —(C═O)(CR₇R₇′)ₙ—ONO₂, —(C═O)(CR₇R₇′)ₘ—CH(NH₂)CH₂ONO₂. In embodiments of the compounds of Formula (I), R₅ is H, halogen, C₁-C₆ alkyl, or —(C—O)(CR₇R₇′)ₘ—CH(NH₂)CH₂ONO₂. In embodiments of the compounds of Formula (I), R₅ is H, halogen, C₁-C₆ alkyl, or —(C═O)(CR₇R₇′)ₙ—ONO₂.

In embodiments of the compounds of Formula (I), R₅ is —(C═O)(CR₇R₇′)ₙ—ONO₂ or —(C═O)(CR₇R₇′)ₘ—CH(NH₂)CH₂ONO₂. In embodiments of the compounds of Formula (I), R₅ is —(C═O)(CR₇R₇′)ₙ—ONO₂. In embodiments of the compounds of Formula (I), R₅ is —(C═O)(CR₇R₇′)ₘ—CH(NH₂)CH₂ONO₂.

In embodiments of the compounds of Formula (I), R₇ and R₇′ are independently H or C₁-C₆ alkyl. In embodiments of the compounds of Formula (I), R₇ and R₇′ are independently H or C₁-C₃ alkyl. In embodiments of the compounds of Formula (I), R₇ and R₇′ are independently H or halogen alkyl. In embodiments of the compounds of Formula (I), R₇ and R₇′ are independently halogen or C₁-C₆ alkyl. In embodiments of the compounds of Formula (I), R₇ and R₇′ are independently halogen or C₁-C₃ alkyl.

In embodiments of the compounds of Formula (I), X is O, S, NH and —O—(P═O)OHO—. In embodiments of the compounds of Formula (I), X is absent, S, NH and —O—(P═O)OHO—. In embodiments of the compounds of Formula (I), X is absent, O, S or —O—(P═O)OHO—. In embodiments of the compounds of Formula (I), X is absent, O, S, or NH. In embodiments of the compounds of Formula (I), X is O or —O—(P═O)OHO—.

In embodiments of the compounds of Formula (I), X is absent. In embodiments of the compounds of Formula (I), X is O. In embodiments of the compounds of Formula (I), X is S. In embodiments of the compounds of Formula (I), X is NH. In embodiments of the compounds of Formula (I), X is —O—(P═O)OHO—.

In embodiments of the compounds of Formula (I), Y is O, S, NH and —O—(P═O)OHO—. In embodiments of the compounds of Formula (I), Y is absent, S, NH and —O—(P═O)OHO—. In embodiments of the compounds of Formula (I), Y is absent, O, S or —O—(P═O)OHO—. In embodiments of the compounds of Formula (I), Y is absent, O, S, or NH. In embodiments of the compounds of Formula (I), Y is O or —O—(P═O)OHO—.

In embodiments of the compounds of Formula (I), Y is absent. In embodiments of the compounds of Formula (I), Y is O. In embodiments of the compounds of Formula (I), Y is S. In embodiments of the compounds of Formula (I), Y is NH. In embodiments of the compounds of Formula (I), Y is —O—(P═O)OHO—.

In embodiments of the compounds of Formula (I), Z is O, S, NH and —O—(P═O)OHO—. In embodiments of the compounds of Formula (I), Z is absent, S, NH and —O—(P═O)OHO—. In embodiments of the compounds of Formula (I), Z is absent, O, S or —O—(P═O)OHO—. In embodiments of the compounds of Formula (I), Z is absent, O, S, or NH. In embodiments of the compounds of Formula (I), Z is O or —O—(P═O)OHO—.

In embodiments of the compounds of Formula (I), Z is absent. In embodiments of the compounds of Formula (I), Z is O. In embodiments of the compounds of Formula (I), Z is S. In embodiments of the compounds of Formula (I), Z is NH. In embodiments of the compounds of Formula (I), Z is —O—(P═O)OHO—.

In embodiments of the compounds of Formula (I), nis 1. In embodiments of the compounds of Formula (I), n is 2. In embodiments of the compounds of Formula (I), n is 3. In embodiments of the compounds of Formula (I), n is 4. In embodiments of the compounds of Formula (I), n is 5.

In embodiments of the compounds of Formula (I), m is 2. In embodiments of the compounds of Formula (I), m is 3. In embodiments of the compounds of Formula (I), m is 4. In embodiments of the compounds of Formula (I), m is 5.

In embodiments of the compounds of Formula (I), R₃ is H and X is absent. In embodiments of compounds of Formula (I), R₃ is halogen and X is absent. In embodiments of compounds of Formula (I), R₃ is H and X is O. In embodiments of compounds of Formula (I), R₃ is C₁-C₆ alkyl and X is O. In embodiments of compounds of Formula (I), R₃ is C₁, F, Br, or I and X is absent. In embodiments of compounds of Formula (I), R₃ is F and X is absent.

In embodiments of the compounds of Formula (I), R₄ is H and Y is absent. In embodiments of compounds of Formula (I), R₄ is halogen and Y is absent. In embodiments of compounds of Formula (I), R₄ is H and Y is O. In embodiments of compounds of Formula (II), R₄ is C₁-C₆ alkyl and Y is O. In embodiments of compounds of Formula (I), R₄ is C₁, F, Br, or I and Y is absent. In embodiments of compounds of Formula (I), R₄ is F and Y is absent.

In embodiments of the compounds of Formula (I), R₅ is H and Z is absent. In embodiments of compounds of Formula (I), R₅ is halogen and Z is absent. In embodiments of compounds of Formula (I), R₅ is H and Z is O. In embodiments of compounds of Formula (I), R₅ is C₁-C₆ alkyl and Z is O. In embodiments of compounds of Formula (I), R₅ is C₁, F, Br, or I and Z is absent. In embodiments of compounds of Formula (II), R₅ is F and Z is absent.

### Formula (II):

In embodiments, provided herein is a compound of Formula (II):

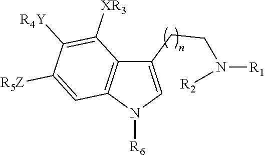

or a pharmaceutically acceptable salt thereof; wherein,
R₃, R₄, and R₅ are independently H, halogen, C₁-C₆ alkyl, —(C—O)(CR₇R₇′)ₙ—ONO₂, or —(C—O)(CR₇R₇′)ₘ—CH(NH₂)CH₂ONO₂;
R₁ and R₆ are independently H, C₁-C₆ alkyl, —(C═O)(CR₇R₇′)ₙ—ONO₂, or —(C—O)(CR₇R₇′)ₘ—CH(NH₂)CH₂ONO₂;
R₂ is H or C₁-C₆ alkyl; and
R₇ and R₇′ are independently H, halogen, or C₁-C₆ alkyl;
X, Y, and Z are independently absent, O, S, NH and —O—(P═O)OHO—,
m is 2, 3, or 4; and
each n is independently 1, 2, 3, 4 or 5;
wherein at least one of R₁, R₃, R₄, R₅ and R₆ is —(C═O)(CR₇R₇′)ₙ—ONO₂, —(C—O)(CR₇R₇′)ₘ—CH(NH₂)CH₂ONO₂.

In embodiments of the compounds of Formula (II), R₁ is C₁-C₆ alkyl. In embodiments of the compounds of Formula (II), R₁ is methyl, ethyl, or propyl. In embodiments of the compounds of Formula (II), R₁ is methyl. In embodiments of the compounds of Formula (II), R₁ is H.

In embodiments of the compounds of Formula (II), R₁ is —(C═O)(CR₇R₇′)ₙ—ONO₂.

In embodiments of the compounds of Formula (II), R₁ is —(C—O)(CR₇R₇′)ₘ—CH(NH₂)CH₂ONO₂. In embodiments of the compounds of Formula (II), R₁ is —(C—O)(CH₂)ₙ—ONO₂. In embodiments of the compounds of Formula (II), R₁ is —(C═O)(CH₂)ₘ—CH(NH₂)CH₂ONO₂. In embodiments of the compounds of Formula (II), R₁ is —(C—O)(CH₂)₃—ONO₂. In embodiments of the compounds of Formula (II), R₁ is —(C—O)(CH₂) 2-CH(NH₂)CH₂ONO₂.

In embodiments of the compounds of Formula (II), R₁ is C₁-C₆ alkyl, —(C═O)(CR₇R₇′)ₙ—ONO₂, or —(C═O)(CR₇R₇′)ₘ—CH(NH₂)CH₂ONO₂. In embodiments of the compounds of Formula (II), R₁ is H, —(C═O)(CR₇R₇′)ₙ—ONO₂, or —(C—O)(CR₇R₇′)ₘ—CH(NH₂)CH₂ONO₂. In embodiments of the compounds of Formula (II), R₁ is H, C₁-C₆ alkyl, or —(C—O)(CR₇R₇′)ₘ—CH(NH₂)CH₂ONO₂. In embodiments of the compounds of Formula (II), R₁ is H, C₁-C₆ alkyl, or —(C═O)(CR₇R₇′)ₙ—ONO₂. In embodiments of the compounds of Formula (II), R₁ is —(C═O)(CR₇R₇′)ₙ—ONO₂ or —(C═O)(CR₇R₇′)ₘ—CH(NH₂)CH₂ONO₂. In embodiments of the compounds of Formula (II), R₁ is C₁-C₆ alkyl or —(C═O)(CR₇R₇′)ₘ—CH(NH₂)CH₂ONO₂. In embodiments of the compounds of Formula (II), R₁ is C₁-C₆ alkyl, or —(C═O) (is halogen, C₁-C₆ alkyl, —(C═O)(CR₇R₇′)ₙ—ONO₂, —(C═O)(CR₇R₇′)ₘ—CH(NH₂)CH₂ONO₂.

In embodiments of the compounds of Formula (II), R₃ is H, halogen, or C₁-C₆ alkyl. In embodiments of the compounds of Formula (II), R₃ is H or halogen. In embodiments of the compounds of Formula (II), R₃ is H or C₁-C₆ alkyl. In embodiments of the compounds of Formula (II), R₃ is halogen or C₁-C₆ alkyl. In embodiments of the compounds of Formula (II), R₃ is H. In embodiments of the compounds of Formula (II), R₃ is halogen. In embodiments of the compounds of Formula (II), R₃ is C₁-C₆ alkyl.

In embodiments of the compounds of Formula (II), R₃ is H, C₁-C₆ alkyl, —(C═O)(CR₇R₇′)ₙ—ONO₂, —(C═O)(CR₇R₇′)ₘ—CH(NH₂)CH₂ONO₂. In embodiments of the compounds of Formula (II), R₃ is H, halogen, —(C═O)(CR₇R₇′)ₙ—ONO₂, —(C═O)(CR₇R₇′)ₘ—CH(NH₂)CH₂ONO₂.

In embodiments of the compounds of Formula (II), R₃ is H, halogen, C₁-C₆ alkyl, or —(C═O)(CR₇R₇′)ₘ—CH(NH₂)CH₂ONO₂. In embodiments of the compounds of Formula (II), R₃ is H, halogen, C₁-C₆ alkyl, or —(C═O)(CR₇R₇′)ₙ—ONO₂. In embodiments of the compounds of Formula (II), R₃ is —(C═O)(CR₇R₇′)ₙ—ONO₂ or —(C—O)(CR₇R₇′)ₘ—CH(NH₂)CH₂ONO₂. In embodiments of the compounds of Formula (II), R₃ is —(C—O)(CR₇R₇′)ₙ—ONO₂. In embodiments of the compounds of Formula (II), R₃ is —(C═O)(CR₇R₇′)ₘ—CH(NH₂)CH₂ONO₂.

In embodiments of the compounds of Formula (II), R₄ is H, halogen, or C₁-C₆ alkyl. In embodiments of the compounds of Formula (II), R₄ is H or halogen. In embodiments of the compounds of Formula (II), R₁ is H or C₁-C₆ alkyl. In embodiments of the compounds of Formula (II), R₄ is halogen or C₁-C₆ alkyl.

In embodiments of the compounds of Formula (II), R₄ is halogen, C₁-C₆ alkyl, —(C═O)(CR₇R₇′)ₙ—ONO₂, —(C═O)(CR₇R₇′)ₘ—CH(NH₂)CH₂ONO₂. In embodiments of the compounds of Formula (II), R₄ is H, C₁-C₆ alkyl, —(C═O)(CR₇R₇′)ₙ—ONO₂, —(C═O)(CR₇R₇′)ₘ—CH(NH₂)CH₂ONO₂. In embodiments of the compounds of Formula (II), R₄ is H, halogen, —(C═O)(CR₇R₇′)ₙ—ONO₂, —(C═O)(CR₇R₇′)ₘ—CH(NH₂)CH₂ONO₂. In embodiments of the compounds of Formula (II), R₄ is H, halogen, C₁-C₆ alkyl, or —(C—O)(CR₇R₇′)ₘ—CH(NH₂)CH₂ONO₂. In embodiments of the compounds of Formula (II), R₄ is H, halogen, C₁-C₆ alkyl, or —(C—O)(CR₇R₇′)ₙ—ONO₂. In embodiments of the compounds of Formula (II), R₄ is —(C═O)(CR₇R₇′)ₙ—ONO₂ or —(C═O)(CR₇R₇′)ₘ—CH(NH₂)CH₂ONO₂. In embodiments of the compounds of Formula (II), R₄ is —(C═O)(CR₇R₇′)ₙ—ONO₂. In embodiments of the compounds of Formula (II), R₄ is —(C═O)(CR₇R₇′)ₘ—CH(NH₂)CH₂ONO₂.

In embodiments of the compounds of Formula (II), R₅ is H, halogen, or C₁-C₆ alkyl. In embodiments of the compounds of Formula (II), R₅ is H or halogen. In embodiments of the compounds of Formula (II), R₅ is H or C₁-C₆ alkyl. In embodiments of the compounds of Formula (II), R₅ is halogen or C₁-C₆ alkyl. In embodiments of the compounds of Formula (II), R₅ is H. In embodiments of the compounds of Formula (II), R₅ is halogen. In embodiments of the compounds of Formula (II), R₅ is C₁-C₆ alkyl.

In embodiments of the compounds of Formula (II), R₅ is halogen, C₁-C₆ alkyl, —(C═O)(CR₇R₇′)ₙ—ONO₂, —(C═O)(CR₇R₇′)ₘ—CH(NH₂)CH₂ONO₂. In embodiments of the compounds of Formula (II), R₅ is H, C₁-C₆ alkyl, —(C═O)(CR₇R₇′)ₙ—ONO₂, —(C═O)(CR₇R₇′)ₘ—CH(NH₂)CH₂ONO₂. In embodiments of the compounds of Formula (II), R₅ is H, halogen, —(C—O)(CR₇R₇′)ₙ—ONO₂, —(C═O)(CR₇R₇′)ₘ—CH(NH₂)CH₂ONO₂. In embodiments of the compounds of Formula (II), R₅ is H, halogen, C₁-C₆ alkyl, or —(C═O)(CR₇R₇′)ₘ—CH(NH₂)CH₂ONO₂. In embodiments of the compounds of Formula (II), R₅ is H, halogen, C₁-C₆ alkyl, or —(C═O)(CR₇R₇′)ₙ—ONO₂. In embodiments of the compounds of Formula (II), R₅ is —(C═O)(CR₇R₇′)ₙ—ONO₂ or —(C═O)(CR₇R₇′)ₘ—CH(NH₂)CH₂ONO₂. In embodiments of the compounds of Formula (II), R₅ is —(C═O)(CR₇R₇′)ₙ—ONO₂. In embodiments of the compounds of Formula (II), R₅ is —(C═O)(CR₇R₇′)ₘ—CH(NH₂)CH₂ONO₂.

In embodiments of the compounds of Formula (II), R₆ is H or C₁-C₆ alkyl. In embodiments of the compounds of Formula (II), R₆ is C₁-C₆ alkyl. In embodiments of the compounds of Formula (II), R₆ is methyl, ethyl, or propyl. In embodiments of the compounds of Formula (II), R₆ is methyl. In embodiments of the compounds of Formula (II), R₆ is H.

In embodiments of the compounds of Formula (II), R₆ is —(C═O)(CR₇R₇′)ₙ—ONO₂.

In embodiments of the compounds of Formula (II), R₆ is —(C—O)(CR₇R₇′)ₘ—CH(NH₂)CH₂ONO₂. In embodiments of the compounds of Formula (II), R₆ is —(C—O)(CH₂)ₙ—ONO₂. In embodiments of the compounds of Formula (II), R₆ is —(C═O)(CH₂)ₘ—CH(NH₂)CH₂ONO₂. In embodiments of the compounds of Formula (II), R₆ is —(C═O)(CH₂)₃—ONO₂. In embodiments of the compounds of Formula (II), R₆ is —(C═O)(CH₂)₂—CH(NH₂)CH₂ONO₂. In embodiments of the compounds of Formula (II), R₆ is C₁-C₆ alkyl, —(C═O)(CR₇R₇′)ₙ—ONO₂, or —(C═O)(CR₇R₇′)ₘ—CH(NH₂)CH₂ONO₂. In embodiments of the compounds of Formula (II), R₆ is H, —(C—O)(CR₇R₇′)ₙ—ONO₂, or —(C═O)(CR₇R₇′)ₘ—CH(NH₂)CH₂ONO₂. In embodiments of the compounds of Formula (II), R₆ is H, C₁-C₆ alkyl, or —(C—O)(CR₇R₇′)ₘ—CH(NH₂)CH₂ONO₂. In embodiments of the compounds of Formula (II), R₆ is H, C₁-C₆ alkyl, or —(C═O)(CR₇R₇′)ₙ—ONO₂. In embodiments of the compounds of Formula (II), R₆ is —(C—O)(CR₇R₇′)ₙ—ONO₂ or —(C═O)(CR₇R₇′)ₘ—CH(NH₂)CH₂ONO₂. In embodiments of the compounds of Formula (II), R₆ is C₁-C₆ alkyl or —(C═O)(CR₇R₇′)ₘ—CH(NH₂)CH₂ONO₂.

In embodiments of the compounds of Formula (II), R₆ is C₁-C₆ alkyl, or —(C═O)(CR₇R₇′)ₙ—ONO₂. In embodiments of the compounds of Formula (II), R₆ is H or —(C═O)(CR₇R₇′)ₙ—ONO₂. In embodiments of the compounds of Formula (II), R₆ is H or —(C═O)(CR₇R₇′)ₘ—CH(NH₂)CH₂ONO₂.

In embodiments of the compounds of Formula (II), R₇ and R₇′ are independently H or C₁-C₆ alkyl. In embodiments of the compounds of Formula (II), R₇ and R₇′ are independently H or C₁-C₃ alkyl. In embodiments of the compounds of Formula (II), R₇ and R₇′ are independently H or halogen alkyl. In embodiments of the compounds of Formula (II), R₇ and R₇′ are independently halogen or C₁-C₆ alkyl. In embodiments of the compounds of Formula (II), R₇ and R₇′ are independently halogen or C₁-C₃ alkyl.

In embodiments of the compounds of Formula (II), X is O, S, NH and —O—(P═O)OHO—. In embodiments of the compounds of Formula (II), X is absent, S, NH and —O—(P═O)OHO—. In embodiments of the compounds of Formula (II), X is absent, O, S or —O—(P═O)OHO—. In embodiments of the compounds of Formula (II), X is absent, O, S, or NH. In embodiments of the compounds of Formula (II), X is absent. In embodiments of the compounds of Formula (II), X is O. In embodiments of the compounds of Formula (II), X is S. In embodiments of the compounds of Formula (II), X is NH. In embodiments of the compounds of Formula (II), X is —O—(P═O)OHO—. In embodiments of the compounds of Formula (II), X is O or —O—(P═O)OHO—.

In embodiments of the compounds of Formula (II), Y is O, S, NH and —O—(P═O)OHO—. In embodiments of the compounds of Formula (II), Y is absent, S, NH and —O—(P═O)OHO—. In embodiments of the compounds of Formula (II), Y is absent, O, S or —O—(P═O)OHO—. In embodiments of the compounds of Formula (II), Y is absent, O, S, or NH. In embodiments of the compounds of Formula (II), Y is absent. In embodiments of the compounds of Formula (II), Y is O. In embodiments of the compounds of Formula (II), Y is S. In embodiments of the compounds of Formula (II), Y is NH. In embodiments of the compounds of Formula (II), Y is —O—(P═O)OHO—. In embodiments of the compounds of Formula (II), Y is O or —O—(P═O)OHO—.

In embodiments of the compounds of Formula (II), Z is O, S, NH and —O—(P═O)OHO—. In embodiments of the compounds of Formula (II), Z is absent, S, NH and —O—(P═O)OHO—. In embodiments of the compounds of Formula (II), Z is absent, O, S or —O—(P═O)OHO—. In embodiments of the compounds of Formula (II), Z is absent, O, S, or NH. In embodiments of the compounds of Formula (II), Z is absent. In embodiments of the compounds of Formula (II), Z is O. In embodiments of the compounds of Formula (II), Z is S. In embodiments of the compounds of Formula (II), Z is NH. In embodiments of the compounds of Formula (II), Z is —O—(P═O)OHO—. In embodiments of the compounds of Formula (II), Z is O or —O—(P═O)OHO—.

In embodiments of the compounds of Formula (II), n is 1. In embodiments of the compounds of Formula (II), n is 2. In embodiments of the compounds of Formula (II), n is 3. In embodiments of the compounds of Formula (II), n is 4. In embodiments of the compounds of Formula (II), n is 5.

In embodiments of the compounds of Formula (II), m is 2. In embodiments of the compounds of Formula (II), m is 3. In embodiments of the compounds of Formula (II), m is 4. In embodiments of the compounds of Formula (II), m is 5.

In embodiments of the compounds of Formula (II), R₃ is H and X is absent. In embodiments of compounds of Formula (II), R₃ is halogen and X is absent. In embodiments of compounds of Formula (II), R₃ is H and X is O. In embodiments of compounds of Formula (II), R₃ is C₁-C₆ alkyl and X is O. In embodiments of compounds of Formula (II), R₃ is C₁, F, Br, or I and X is absent. In embodiments of compounds of Formula (II), R₃ is F and X is absent.

In embodiments of the compounds of Formula (II), R₄ is H and Y is absent. In embodiments of compounds of Formula (II), R₄ is halogen and Y is absent. In embodiments of compounds of Formula (II), R₄ is H and Y is O. In embodiments of compounds of Formula (II), R₄ is C₁-C₆ alkyl and Y is O. In embodiments of compounds of Formula (II), R₄ is C₁, F, Br, or I and Y is absent. In embodiments of compounds of Formula (II), R₄ is F and Y is absent.

In embodiments of the compounds of Formula (II), R₅ is H and Z is absent. In embodiments of compounds of Formula (II), R₅ is halogen and Z is absent. In embodiments of compounds of Formula (II), R₅ is H and Z is O. In embodiments of compounds of Formula (II), R₅ is C₁-C₆ alkyl and Z is O. In embodiments of compounds of Formula (II), R₅ is C₁, F, Br, or I and Z is absent.

In embodiments of compounds of Formula (II), R₅ is F and Z is absent.

TABLE 1
Compounds of Formula (II)

| No. | Structure | |
|---|---|---|
| 2-1 | 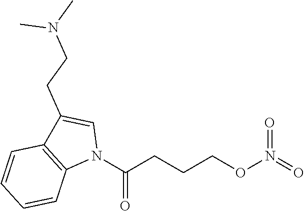 [SMILES: CN(C)CCc1cn(C(=O)CCCO[N+](=O)[O-])c2ccccc12] | 4-(3-(2- (dimethylamino)ethyl)-1H- indol-1-yl)-4-oxobutyl nitrate |
| 2-2 | 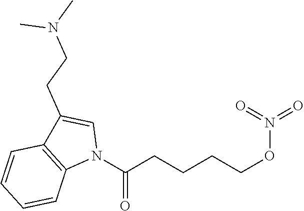 [SMILES: CN(C)CCc1cn(C(=O)CCCCO[N+](=O)[O-])c2ccccc12] | 5-(3-(2- (dimethylamino)ethyl)-1H- indol-1-yl)-5-oxopentyl nitrate |
| 2-3 | 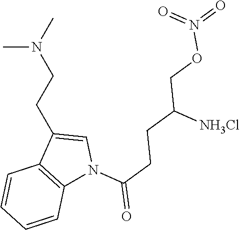 | 2-(chloro-λ⁵-azaneyl)-5-(3- (2-(dimethylamino)ethyl)-1H- indol-1-yl)-5-oxopentyl nitrate |
| 2-4 | 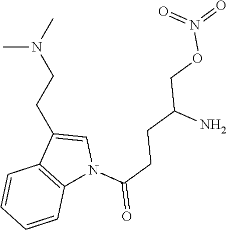 [SMILES: CN(C)CCc1cn(C(=O)CCC(N)CO[N+](=O)[O-])c2ccccc12] | 2-amino-5-(3-(2- (dimethylamino)ethyl)-1H- indol-1-yl)-5-oxopentyl nitrate |
| 2-5 | 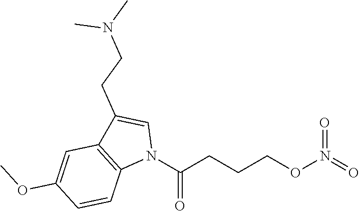 [SMILES: COc1ccc2c(c1)c(CCN(C)C)cn2C(=O)CCCO[N+](=O)[O-]] | 4-(3-(2- (dimethylamino)ethyl)-5- methoxy-1H-indol-1-yl)-4- oxobutyl nitrate |
| 2-6 | 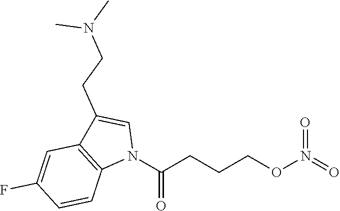 [SMILES: CN(C)CCc1cn(C(=O)CCCO[N+](=O)[O-])c2ccc(F)cc12] | 4-(3-(2- (dimethylamino)ethyl)-5- fluoro-1H-indol-1-yl)-4- oxobutyl nitrate |
| 2-7 | 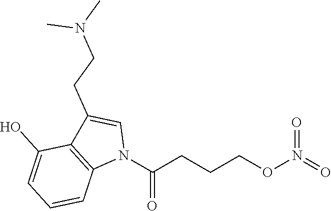 [SMILES: CN(C)CCc1cn(C(=O)CCCO[N+](=O)[O-])c2cccc(O)c12] | 4-(3-(2- (dimethylamino)ethyl)-4- hydroxy-1H-indol-1-yl)-4- oxobutyl nitrate |
| 2-8 | 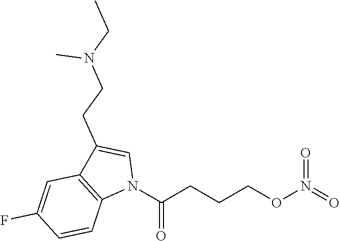 [SMILES: CCN(C)CCc1cn(C(=O)CCCO[N+](=O)[O-])c2ccc(F)cc12] | 4-(3-(2- (ethyl(methyl)amino)ethyl)-5- fluoro-1H-indol-1-yl)-4- oxobutyl nitrate |
| 2-9 | 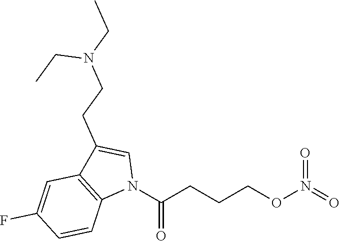 [SMILES: CCN(CC)CCc1cn(C(=O)CCCO[N+](=O)[O-])c2ccc(F)cc12] | 4-(3-(2-(diethylamino)ethyl)- 5-fluoro-1H-indol-1-yl)-4- oxobutyl nitrate |
| 2-10 | 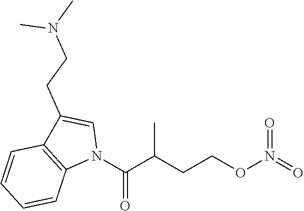 [SMILES: CC(CCO[N+](=O)[O-])C(=O)n1cc(CCN(C)C)c2ccccc21] | 4-(3-(2- (dimethylamino)ethyl)-1H- indol-l-yl)-3-methyl-4- oxobutyl nitrate |
| 2-11 | 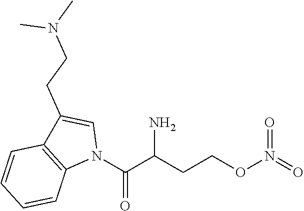 [SMILES: CN(C)CCc1cn(C(=O)C(N)CCO[N+](=O)[O-])c2ccccc12] | 3-amino-4-(3-(2- (dimethylamino)ethyl)-1H- indol-1-yl)-4-oxobutyl nitrate |
| 2-12 | 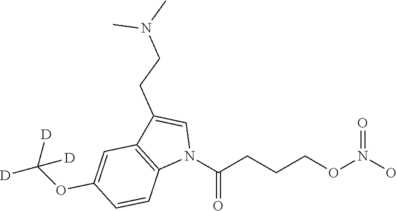 | 4-(3-(2- (dimethylamino)ethyl)-5- (methoxy-d₃)-H-indol-1-yl)- 4-oxobutyl nitrate |
| 2-13 | 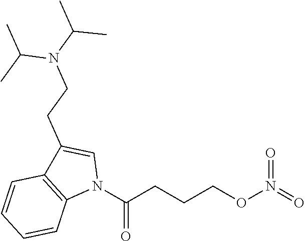 [SMILES: CC(C)N(CCc1cn(C(=O)CCCO[N+](=O)[O-])c2ccccc12)C(C)C] | 4-(3-(2- (diisopropylamino)ethyl)-1H- indol-l-yl)-4-oxobutyl nitrate |
| 2-14 | 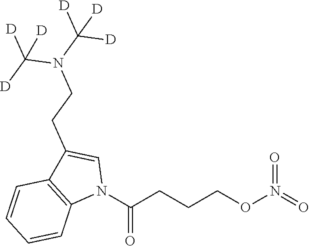 [SMILES: [2H]C([2H])([2H])N(CCc1cn(C(=O)CCCO[N+](=O)[O-])c2ccccc12)C([2H])([2H])[2H]] | 4-(3-(2-(bis(methyl- d₃)amino)ethyl)-1H-indol-1- yl)-4-oxobutyl nitrate |

### Compositions

In embodiments, the present disclosure provides a pharmaceutical composition comprising a therapeutically effective amount of one or more compounds of the present disclosure (e.g., a compound of Formula (I), (II), or Table 1) or a pharmaceutically acceptable salt thereof and a pharmaceutically acceptable excipient.

The pharmaceutically acceptable excipients and adjuvants are added to the composition or formulation for a variety of purposes. In some embodiments, a pharmaceutical composition comprising one or more compounds disclosed herein, or a pharmaceutically acceptable salt thereof, further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

### Methods of Treatment

In one aspect, the present disclosure provides methods of treating a disease or disorder in a subject in need thereof, the methods comprising administering a therapeutically effective amount of a compound described herein (e.g., a compound of Formula (I), (II), or Table 1) or pharmaceutically acceptable salt thereof to the subject.

In embodiments, the disease or disorder is a mental health disease or disorder. In embodiments, the mental health disease or disorder is selected from the group consisting of major depressive disorder, treatment resistant depression, substance use disorders and eating disorders. In embodiments, eating disorders include illnesses such as anorexia nervosa, bulimia nervosa, and other disorders related to eating (e.g., binge eating).

In embodiments, the mental health disease or disorder is an eating disorder.

In embodiments, the mental health disease or disorder is selected from the group consisting of compulsive disorders, anxiety disorders, stress disorders, and rumination.

In embodiments, the mental health disease or disorder is a mood disorder. In embodiments, mood disorders include e.g., depressive disorders, such as major depressive disorder or treatment resistant depression.

In embodiments, the mental health disorder is a substance abuse disorder. In embodiments, substance use related disorders are disorders of maladaptive patterns of substance use, and include criteria, such as recurrent substance use related problems, tolerance to a substance, withdrawal upon discontinuing use, an inability to cut down or control use of the substance, and giving up important social, occupational, or recreational activities because of using the substance. See e.g., the Diagnostic and Statistical Manual of Mental Disorders (DSM-5). In embodiments, the substance use related disorder is a disorder resulting from the use of: alcohol; caffeine; cannabis; hallucinogens (such as phencyclidine or similarly acting arylcyclohexylamines, and other hallucinogens, such as LSD); inhalants; opioids; sedatives, hypnotics, or anxiolytics; stimulants (including amphetamine-type substances, cocaine, and other stimulants); tobacco; and other substances.

In embodiments, administering compounds of the present disclosure (e.g., a compound of Formula (I), (II), or Table 1) or a pharmaceutically acceptable salt thereof releases nitric oxide (NO) (e.g., the compounds of the present disclosure are NO delivery drugs). In embodiments the compounds of the present disclosure are useful for releasing NO in vivo.

### NUMBERED EMBODIMENTS

In addition to the disclosure above, the Examples below, and the appended claims, the disclosure sets for the following numbered embodiments.
1. A compound of Formula (II):

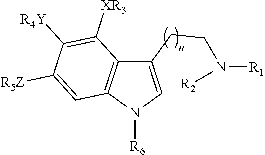

or a pharmaceutically acceptable salt thereof; wherein,
R₃, R₄, and R₅ are independently H, halogen, C₁-C₆ alkyl, —(C═O)(CR₇R₇′)ₙ—ONO₂, or —(C═O)(CR₇R₇′)ₘ—CH(NH₂)CH₂ONO₂;
R₁ and R₆ are independently H, C₁-C₆ alkyl, —(C—O)(CR₇R₇′)ₙ—ONO₂, —(C═O)(CR₇R₇′)ₘ—CH(NH₂)CH₂ONO₂;
R₂ is H or C₁-C₆ alkyl; and
R₇ and R₇′ are independently H, halogen, or C₁-C₆ alkyl;
X, Y, and Z are independently absent, O, S, NH, or —O—(P═O)OHO—,
m is 2, 3, or 4; and
each n is independently 1, 2, 3, 4 or 5;
wherein at least one of R₁, R₃, R₄, R₅ and R₆ is —(C═O)(CR₇R₇′)ₙ—ONO₂, —(C═O)(CR₇R₇′)ₘ—CH(NH₂)CH₂ONO₂.
2. The compound of embodiment 1, wherein Riis C₁-C₆ alkyl.
3. The compound of embodiment 1, wherein R₁ is methyl.
4. The compound of embodiment 1, wherein R₁ is H.
5. The compound of any one of embodiments 1-4, wherein R₂ is C₁-C₆ alkyl.
6. The compound of any one of embodiments 1~4 wherein R₂ is methyl.
7. The compound of any one of embodiments 1-4, wherein R₂ is H.
8. The compound of any one of embodiments 1-7, wherein R₃ is H and X is absent.
9. The compound of any one of embodiments 1-7, wherein R₃ is halogen and X is absent.
10. The compound of any one of embodiments 1-7, wherein R₃ is H and X is O.
11. The compound of any one of embodiments 1-7, wherein R₃ is C₁-C₆ alkyl and X is O.
12. The compound of any one of embodiments 1-11, wherein R₄ is H and Y is absent.
13. The compound of any one of embodiments 1-11, wherein R₄ is H and Y is O.
14. The compound of any one of embodiments 1-11, wherein R₄ is halogen and Y is absent.
15. The compound of any one of embodiments 1-11, wherein R₄ is C₁-C₆ alkyl and Y is O.
16. The compound of any one of embodiments 1-15, wherein R₅ is H and Z is absent.
17. The compound of any one of embodiments 1-15, wherein R₅ is H and Z is O.
18. The compound of any one of embodiments 1-15, wherein R₅ is halogen and Z is absent.
19. The compound of any one of embodiments 1-15, wherein R₅ is C₁-C₆ alkyl and Z is absent.
20. The compound of any one of embodiments 1-15, wherein R₅ is C₁-C₆ alkyl and Z is O.
21. The compound of any one of embodiments 1-20, wherein R₆ is —(C—O)(CR₇R₇′)ₙ—ONO₂.
22. The compound of any one of embodiments 1-20, wherein R₆ is —(C═O)(CR₇R₇′)ₘ—CH(NH₂)CH₂ONO₂.
23. The compound of any one of embodiments 1-20, wherein R₆ is —(C═O)(CH₂)ₙ—ONO₂.
24. The compound of any one of embodiments 1-20, wherein R₆ is —(C—O)(CH₂)ₘ—CH(NH₂)CH₂ONO₂.
25. The compound of any one of embodiments 1-20, wherein R₆ is —(C═O)(CH₂)₃—ONO₂.
26. The compound of any one of embodiments 1-25, having the following chemical formula:

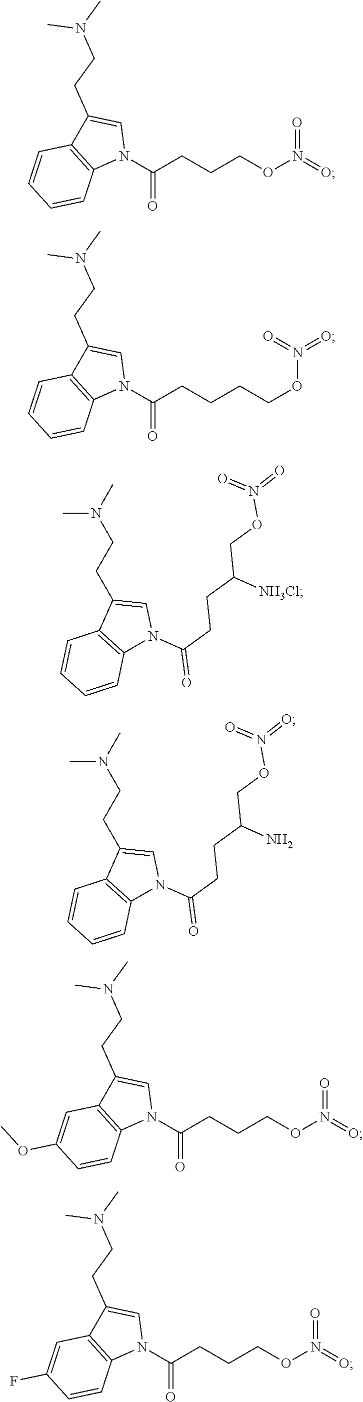

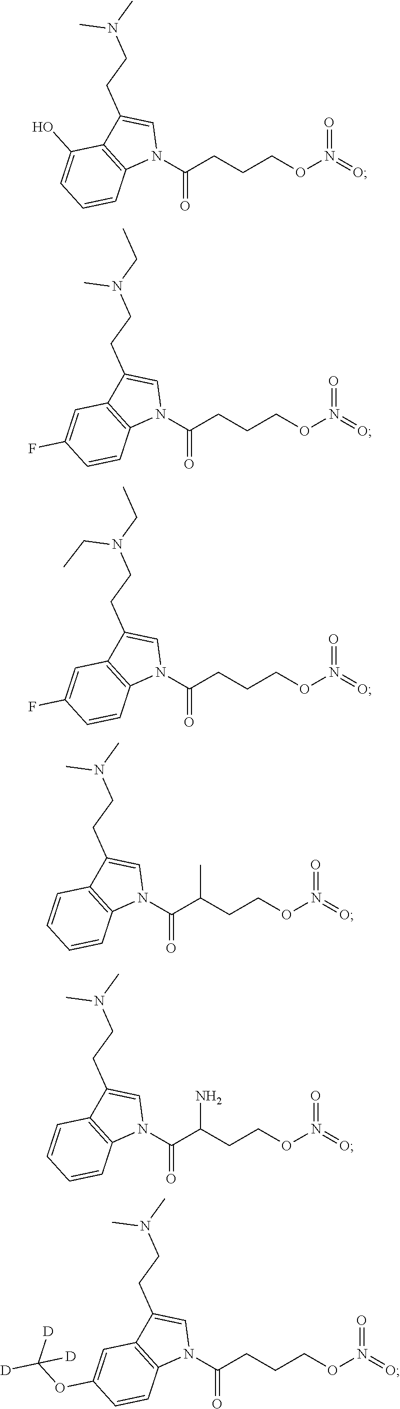

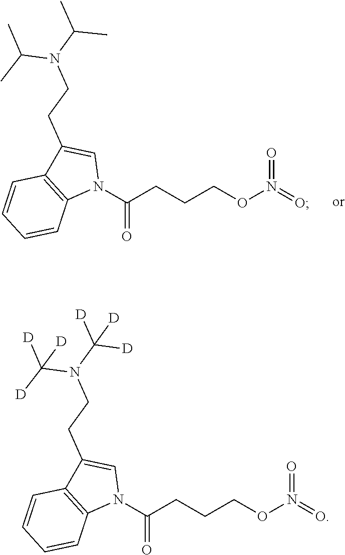 [SMILES: CC(C)N(CCc1cn(C(=O)CCCO[N+](=O)[O-])c2ccccc12)C(C)C ; "or" ; [2H]C([2H])([2H])N(CCc1cn(C(=O)CCCO[N+](=O)[O-])c2ccccc12)C([2H])([2H])[2H]]

27. The compound of embodiment 26, having the following chemical formula:

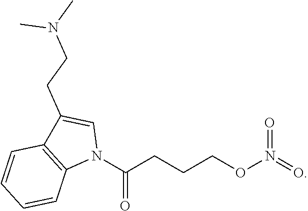 [SMILES: CN(C)CCc1cn(C(=O)CCCO[N+](=O)[O-])c2ccccc12]

28. The compound of embodiment 26, having the following chemical formula:

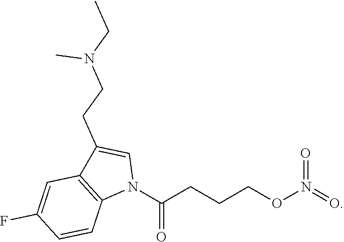 [SMILES: CCN(C)CCc1cn(C(=O)CCCO[N+](=O)[O-])c2ccc(F)cc12]

29. A pharmaceutical composition, comprising a compound of any one of embodiments 1-28 and a pharmaceutically acceptable excipient.
30. A method of treating a mental health disease or disorder, the method comprising administering a therapeutically effective amount of a compound of any one of embodiments 1-28 or pharmaceutical composition of embodiment 29.
31. A compound of Formula (I):

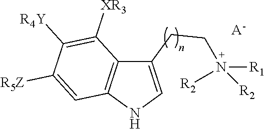

or a pharmaceutically acceptable salt thereof; wherein,
R₁, R₃, R₄ and R₅ are independently H, halogen, C₁-C₆ alkyl, —(C—O)(CR₇R₇′)ₙ—ONO₂, or —(C═O)(CR₇R₇′)ₘ—CH(NH₂)CH₂ONO₂;
R₂ is C₁-C₆ alkyl;
X, Y, and Z are independently absent, H, O, S, NH and —O—(P═O)OHO—;
m is 2, 3, or 4;
each n is independently 1, 2, 3, 4 or 5; and
A⁻ is a pharmaceutically acceptable anion,
wherein at least one of R₁, R₃, R₄ and R₅ are (C═O)(CR₇R₇′)ₙ—ONO₂ or —(C—O)(CR₇R₇′)ₘ—CH(NH₂)CH₂ONO₂.
32. The compound of embodiment 31, wherein R₁ is —(C═O)(CR₇R₇′)ₙ—ONO₂.
33. The compound of embodiment 31, wherein R₁ is —(C═O)(CR₇R₇′)ₘ—CH(NH₂)CH₂ONO₂.
34. The compound of embodiment 31, wherein R₁ is —(C═O)(CH₂)ₙ—ONO₂.
35. The compound of embodiment 31, wherein R₁ is —(C═O)(CH₂)ₘ—CH(NH₂)CH₂ONO₂.
36. The compound of embodiment 31, wherein R₁ is —(C═O)(CH₂)₃—ONO₂.
37. The compound of embodiment 31, wherein R₁ is —(C═O)(CH₂)₂—CH(NH₂)CH₂ONO₂.
38. The compound of any one of embodiments 31-37, wherein R₂ is a C₁-C₃ alkyl.
39. The compound of any one of embodiments 31-38, wherein R₃ is H and X is absent.
40. The compound of any one of embodiments 31-38, wherein R₃ is halogen and X is absent.
41. The compound of any one of embodiments 31-38, wherein R₃ is H and X is O.
42. The compound of any one of embodiments 31-38, wherein R₃ is C₁-C₆ alkyl and X is O.
43. The compound of any one of embodiments 31-42, wherein R₄ is H and Y is absent.
44. The compound of any one of embodiments 31-42, wherein R₄ is H and Y is O.
45. The compound of any one of embodiments 31-42, wherein R₄ is halogen and Y is absent.
46. The compound of any one of embodiments 31-42, wherein R₄ is C₁-C₆ alkyl and Y is O.
47. The compound of any one of embodiments 31-46, wherein R₅ is H and Z is absent.
48. The compound of any one of embodiments 31-46, wherein R₅ is H and Z is O.
49. The compound of any one of embodiments 31-46, wherein R₅ is halogen and Z is absent.
50. The compound of any one of embodiments 31-46, wherein R₅ is C₁-C₆ alkyl and Z is O.
51. A pharmaceutical composition, comprising a compound of any one of embodiments 31-50 and a pharmaceutically acceptable excipient.
52. A method of treating a mental health disease or disorder, the method comprising administering a therapeutically effective amount of a compound of any one of embodiments 31-50 or pharmaceutical composition of embodiment 51.

### EXAMPLES

### Example 1: Methods of Preparing the Compounds of the Present Disclosure

### Synthesis of Compound 2-1

Prodrug 2-1 was synthesized from commercially available intermediate 2-1-1 and 2-1-3 in three steps and described in the Scheme 1.

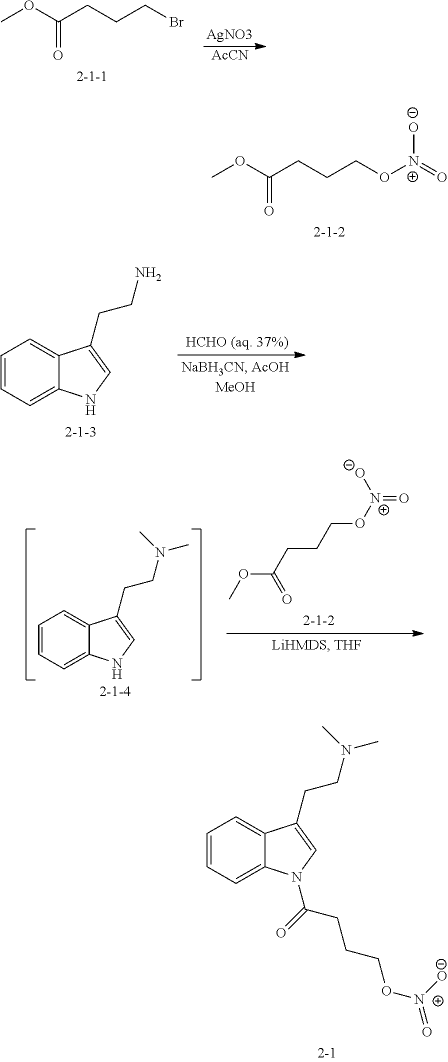

### Synthesis of Intermediate 2-1-1

To a stirred solution of methyl 4-bromobutanoate, 2-1-1 (3 g, 16.5 mmol, 1.0 equiv) in acetonitrile was added AgNO₃ (7.0 g, 41.4 mmol, 2.5 equiv) in portions at room temperature under argon atmosphere. The resulting mixture was stirred for overnight at 80° C. under argon atmosphere. New pot could be detected by TLC. The resulting mixture was filtered, the filter cake was washed with acetonitrile (3×50 mL). The filtrate was concentrated under reduced pressure. The residue was dissolved in ethyl acetate (500 mL). The resulting mixture was washed with 2×500 ml of water, and then the resulting mixture was washed with 3×300 mL of brine. The combined organic layers dried over anhydrous Na₂SO₄. After filtration, the filtrate was concentrated under reduced pressure to afford methyl 4-(nitrooxy)butanoate, 2-1-2 (2.3 g, 85.08%) as a yellow oil. This crude product was used directly for the next step without further purification.

### Synthesis of intermediate 2-1-4 and final Product 1-(3-(2-(dimethylamino)ethyl)-1H-indol-1-yl)-4-(nitrooxy)butanoate (2-1)

To a stirred solution of tryptamine (200 mg, 1.2 mmol, 1.0 equiv) and NaBH₃CN (235.3 mg, 3.7 mmol, 3.0 equiv), AcOH (0.2 mL) in MeOH was added formaldehyde solution (187.4 mg, 6.24 mmol, 5.0 equiv) dropwise at 0° C. under argon atmosphere. The resulting mixture was stirred for overnight at room temperature under argon atmosphere. Desired product could be detected by LCMS. The resulting mixture was filtered, the filter cake was washed with MeOH (3×10 mL). The filtrate was concentrated under reduced pressure. The residue was purified by silica gel column chromatography, eluted with PE/EA (1:1) to afford intermediate as a yellow solid. To a stirred solution of intermediate and methyl 4-(nitrooxy)butanoate (610.8 mg, 3.7 mmol, 3.0 equiv) in THF was added LiHMDS (313.3 mg, 1.8 mmol, 1.5 equiv) in THF dropwise at 0° C. under argon atmosphere. The resulting mixture was stirred for 1 h at 0° C. under argon atmosphere. Desired product could be detected by LCMS. The reaction was quenched with MeOH at 0° C. The resulting mixture was concentrated under vacuum. The residue was purified by reversed-phase flash chromatography with the following conditions: column, C18 silica gel; mobile phase, MeCN in Water (0.1% FA), 5% to 80% gradient in 40 min; detector, UV 254 nm. to afford 25 mg of 1-(3-(2-(dimethylamino)ethyl)-1H-indol-1-yl)-4-(nitrooxy)butanoate (2-1) as a white solid. LCMS of 2-1: [M+H]⁺ 320.10

HNMR-2-1: (400 MHz, DMSO-d₆) δ8.07 (d, J=8.2 Hz, 1H), 7.62 (d, J=7.7 Hz, 1H), 7.52 (s, 1H), 7.36-7.25 (m, 2H), 4.79 (t, J=6.7 Hz, 2H), 4.47 (t, J=6.0 Hz, 2H), 2.80 (t, J=7.5 Hz, 2H), 2.57-2.40 (m, 4H), 2.22 (s, 6H).

1. A compound of Formula (II): 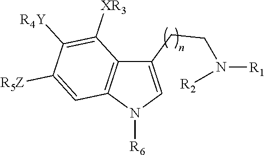
or a pharmaceutically acceptable salt thereof; wherein,
R₃, R₄, and R₅ are independently H, halogen, C₁-C₆ alkyl, —(C—O)(CR₇R₇′)ₙ—ONO₂, or —(C═O)(CR₇R₇′)ₘ—CH(NH₂)CH₂ONO₂;
R₁ and R₆ are independently H, C₁-C₆ alkyl, —(C═O)(CR₇R₇′)ₙ—ONO₂, or —(C—O)(CR₇R₇′)ₘ—CH(NH₂)CH₂ONO₂;
R₂ is C₁-C₆ alkyl; and
R₇ and R₇′ are independently H, halogen, or C₁-C₆ alkyl;
X, Y, and Z are independently absent, O, S, NH, or —O—(P═O)OHO—,
m is 2, 3, or 4; and
each n is independently 1, 2, 3, 4 or 5;
wherein at least one of R₁, R₃, R₄, R₅ and R₆ is —(C═O)(CR₇R₇′)ₙ—ONO₂, or —(C═O)(CR₇R₇′)ₘ—CH(NH₂)CH₂ONO₂.

2. The compound of claim 1, wherein Riis C₁-C₆ alkyl.

3. The compound of claim 1, wherein R₁ is methyl.

4. The compound of claim 1, wherein R₁ is H.

5. (canceled)

6. The compound of claim 1, wherein R₂ is methyl.

7. (canceled)

8. The compound of claim 1, wherein R₃ is H or halogen and X is absent.

9. (canceled)

10. The compound of claim 1, wherein R₃ is H or C₁-C₆ alkyl and X is O.

11. (canceled)

12. The compound of claim 1, wherein R₄ is H or C₁-C₆ alkyl and Y is absent or Q.

13. (canceled)

14. The compound of claim 1, wherein R₄ is halogen and Y is absent.

15. (canceled)

16. The compound of claim 1, wherein R₅ is H and Z is absent or O.

17-18. (canceled)

19. The compound of claim 1, wherein R₅ is C₁-C₆ alkyl or halogen and Z is absent, or R₅ is C₁-C₆ alkyl and Z is Q.

20-28. (canceled)

29. A pharmaceutical composition, comprising a compound of claim 1 and a pharmaceutically acceptable excipient.

30. A method of treating a mental health disease or disorder, the method comprising administering a therapeutically effective amount of a compound of claim 1.

31. A compound of Formula (I): 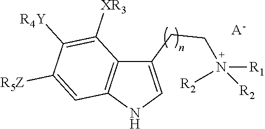
or a pharmaceutically acceptable salt thereof; wherein,
R₁, R₃, R₄ and R₅ are independently H, halogen, C₁-C₆ alkyl, —(C═O)(CR₇R₇′)ₙ—ONO₂, or —(C═O)(CR₇R₇′)ₘ—CH(NH₂)CH₂ONO₂;
each R₂ is independently C₁-C₆ alkyl;
X, Y, and Z are independently absent, H, O, S, NH and —O—(P═O)OHO—;
m is 2, 3, or 4;
each n is independently 1, 2, 3, 4 or 5; and
A⁻ is a pharmaceutically acceptable anion,
wherein at least one of R₁, R₃, R₄ and R₅ are —(C—O)(CR₇R₇′)ₙ—ONO₂ or —(C═O)(CR₇R₇′)ₘ—CH(NH₂)CH₂ONO₂.

32. The compound of claim 31, wherein R₁ is —(C═O)(CR₇R₇′)ₙ—ONO₂ or —(C═O)(CR₇R₇′)ₘ—CH(NH₂)CH₂ONO₂.

33. (canceled)

34. The compound of claim 31, wherein R₁ is —(C═O)(CH₂)ₙ—ONO₂, —(C═O)(CH₂)ₘ—CH(NH₂)CH₂ONO₂, —(CO)(CH₂)₃—ONO₂, or —(C═O)(CH₂)₂—CH(NH₂)CH₂ONO₂.

35-37. (canceled)

38. The compound of claim 31, wherein R₂ is a C₁-C₃ alkyl.

39. The compound of claim 31, wherein R₃ is H or halogen and X is absent.

40. (canceled)

41. The compound of claim 31, wherein R₃ is H or C₁-C₆ alkyl and X is O.

42. (canceled)

43. The compound of claim 31, wherein R₄ is H and Y is absent or O.

44. (canceled)

45. The compound of claim 31, wherein R₄ is halogen and Y is absent, or R₄ is C₁-C₆ alkyl and Y is O.

46. (canceled)

47. The compound of claim 31, wherein R₅ is H or C₁-C₆ alkyl and Z is absent or O.

48. (canceled)

49. The compound of claim 31, wherein R₅ is halogen and Z is absent.

50. (canceled)

51. A pharmaceutical composition, comprising a compound of claim 31 and a pharmaceutically acceptable excipient.

52. A method of treating a mental health disease or disorder, the method comprising administering a therapeutically effective amount of a compound of claim 31.
